(19) United States
(12) Patent Application Publication
(10) Pub. No.: US 2021/0154363 A1
(43) Pub. Date: May 27, 2021

(54) COLLAGEN MELANOCYTE COMPLEX, PREPARATION METHOD AND USE THEREOF

(71) Applicant: Hangzhou Singclean Medical Products Co., Ltd, Hangzhou (CN)

(72) Inventors: Jin Zeng, Hangzhou (CN); Caili Ma, Hangzhou (CN); Weiqing Sun, Hangzhou (CN); Wei Huang, Hangzhou (CN)

(21) Appl. No.: 16/834,220

(22) Filed: Mar. 30, 2020

(30) Foreign Application Priority Data
Nov. 25, 2019 (CN) 201911161955.4

(51) Int. Cl. A61L 27/24; A61L 27/36

(52) U.S. Cl. CPC A61L 27/24; A61L 27/362

(57) ABSTRACT

The present disclosure provides a preparation method of a collagen melanocyte complex and use of the collagen melanocyte complex. The collagen material is a scaffold material with good biocompatibility, mechanical properties, and degradation performance, which is prepared based on tissue engineering technology. The present disclosure relates to the preparation of a collagen scaffold and the construction of a collagen melanocyte complex. Melanocytes on the scaffold material have higher cell activity and better melanin secretion ability. The collagen melanocyte complex avoids the loss of cell suspension during cell transplantation and provides a good three-dimensional growth environment for cells. The collagen melanocyte complex prepared by the present disclosure can be used in patients with vitiligo or patients with pigment deficiency and epidermal damage.

### CROSS-REFERENCE TO RELATED APPLICATION

This application claims priority of Chinese Patent Application No. 201911161955.4 filed on Nov. 25, 2019, the entire content of which is incorporated herein by reference.

### FIELD OF THE DISCLOSURE

The present disclosure relates to biomedical filed, and in particular, to a collagen melanocyte complex, a preparation method and a use thereof.

### BACKGROUND OF THE DISCLOSURE

Vitiligo is a common primary, localized or widespread, depigmented skin mucosal disease. One or several hypopigmented spots occur at the incipience of the skin lesion, and gradually develop into a clear milky white patch. The boundaries of the white patches are clear, and the hairs inside the white patches are normal or turn white. The disease can occur in all parts of the body, common in the back of fingers, wrists, forearms, faces, necks and around the genitals. The disease can occur at any age, and there is no obvious gender difference. It is usually divided into localized-type, scattered-type and pan-type. The disease damages the patient's skin, affects the appearance, and then affects the patient's normal life, work and social life, and reduces the quality of life. It is one of the dermatologically intractable and incurable diseases.

Traditional treatments for vitiligo include oral medications, UV radiation, and surgeries. UV radiation treatments are quite limited in cures, and medication treatments have large side effects, which are both not suitable for long-term use. Currently, surgical treatments mainly include autologous epidermal transplantation, but the limitation is that epidermal transplantation requires the removal of skin at normal sites, resulting in new wounds and difficult nursing. At present, the cell suspension used in clinical cell transplantation is easy to lose, and is not suitable for large-area transplantation. In addition, it is difficult to fix at the tip. Therefore, using tissue engineering technology to construct melanocyte-scaffold composites can effectively improve the shortcomings of the above treatments. This kind of composite material plays a fixed supporting role for cells, avoiding the loss of cell suspension. At the same time, the scaffold materials can recruit more repair cells, which secrete endogenous repair factors to participate in tissue reconstruction and wound repair.

### SUMMARY OF THE DISCLOSURE

The purpose of the present disclosure is to provide a collagen melanocyte complex, a preparation method and a use thereof, so as to overcome the deficiencies in the prior art.

According to a first aspect of the present disclosure, the present disclosure adopts the following technical solution:

a method for preparing a collagen melanocyte complex, which is characterized in preparing a cross-linked collagen sponge scaffold material firstly, inoculating melanocytes on the cross-linked collagen sponge scaffold material, and then culturing in vitro.

A method for preparing the cross-linked collagen sponge scaffold material further comprises the following steps: adding a collagen with appropriate size to a reaction solvent, adding a cross-linking agent to the reaction solvent, and magnetically stirring; after the completion of reaction, performing washing, freeze-drying, and sterilizing.

Preferably, the collagen is aquatic animal collagen, such as fish scale collagen, fish skin collagen, and the like, or mammalian collagen, such as bovine Achilles tendon collagen, pig skin collagen, and the like. The collagen has a thickness of 0.5-10 mm sponge, which is cut into a shape not larger than a petri dish, such as a rectangle or square with a side length of 0.5-3 cm. The reaction solvent is ethanol or water phase, preferably 90% ethanol or PBS solution with pH=5.5. The cross-linking agent is EDC and NHS, and a molar ratio of EDC:NHS is in a range of 1:1 to 1:4. A time of the magnetic stirring is in a range of 2-72 h, preferably in a range of 24-48 h. A reaction temperature is in a range of 4-30° C., preferably in a range of 20-25° C. The washing condition includes soaking in phosphate buffer for 1-2 h, and washing in purified water for 1-24 h. A temperature of the freeze-drying can be in a range of −60 to −80° C.

Inoculating melanocytes on the cross-linked collagen sponge scaffold material and culturing in vitro comprise the following steps: pre-plating the collagen sponge scaffold material onto a cell culture plate, inoculating 100 μl-1000 μl melanocytes to the collagen sponge scaffold material at a density of 2*10⁴ to 2*10⁷ cells/ml, and then culturing in a cell incubator under the condition of 37° C., 5% CO₂, and saturated humidity; after the collagen scaffold inoculated with melanocytes is cultured for 30 min-2 h, supplementing with cells medium 1 ml-10 ml, and continuing culturing in a cell incubator under the condition of 37° C., 5% CO₂, and saturated humidity;

replacing the cell medium of the culture plate covered with the collagen sponge scaffold material with a new cell medium every two days, a culture time being in a range of 1 to 25 days, and obtaining the melanocyte-collagen complex.

The melanocytes are human melanocytes. The human melanocytes are melanocytes extracted from autologous or allogeneic tissues. The autologous or allogeneic tissue can be skin, hair follicles, and the like.

According to a second aspect of the present disclosure, it provides a collagen melanocyte complex prepared by the above technical solution.

According to a third aspect of the present disclosure, it provides a use of the above collagen melanocyte complex for preparing biological materials for treating patients with pigment deficiency and epidermal damage.

When constructing a collagen melanocyte complex, skin color can be adjusted by adjusting the density, number, or culture time of inoculated cells.

In summary, the present disclosure provides a method for preparing a collagen melanocyte complex, and a collagen melanocyte complex prepared by the method. The method and the complex provide a new treatment approach for patients with vitiligo and have potential application prospects in the biomedical field.

The beneficial effects of the present disclosure are as mentioned below. By visual observation (FIG. 1, FIG. 3) and microscopic observation (FIG. 2) of the cultured constructed collagen melanocyte composite material, it is found that this material is suitable for the growth of melanocyte and can secrete melanin normally. It can adjust the skin color by different densities and culture days of the inoculated cells. Furthermore, it is found that the cross-linked collagen scaffold (FIG. 1) has a much better anti-degradation ability than the uncross-linked collagen scaffold (FIG. 3). The cross-linked collagen scaffold has good mechanical properties and can meet the requirements of removing, moving, and transplanting from the cell culture plate to the wound without breaking. At the same time, the composite material prepared by this method avoids a large amount of cell loss in the short term caused by only cell transplantation on the one hand. On the other hand, the scaffold can be used as a cell carrier, which has better cell compatibility. It can activate endogenous repair factors, and recruit more biologically active factors participating in tissue reconstruction, thereby exerting better results. The collagen scaffold of the present disclosure has advantages of easy-to-obtain raw materials, simple preparation of the scaffold, excellent mechanical properties and anti-degradation properties of the constructed composite material in the early stage. After the collagen scaffold transplanted to the wound surface in the later stage, while achieving the tissue repair, the scaffold can fall off or degrade automatically. A new clinical treatment approach is provided for patients with vitiligo.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows microscopic observation of morphology of melanocytes cultured with cross-linked collagen scaffold extract.

FIG. 2 is a picture of laser confocal microscope observation of the collagen melanocyte complex of Example 4 on day 15.

FIG. 3 shows the pictures of visual observation of melanocyte proliferation and melanin secretion; in which a-1 is the picture of blank group took after culturing in vitro for 6 days, a-2 is the picture of blank group took after culturing in vitro for 15 days, b-1 is the picture of uncross-linked group of Example 5 took after culturing in vitro for 6 days, b-2 is the picture of uncross-linked group of Example 5 took after culturing in vitro for 15 days, c-1 is the picture of cross-linked group of Example 1 took after culturing in vitro for 6 days, c-2 is the picture of cross-linked group of Example 1 took after culturing in vitro for 11 days, d-1 is the picture of cross-linked group of Example 2 took after culturing in vitro for 6 days, d-2 is the picture of cross-linked group of Example 2 took after culturing in vitro for 15 days, e-1 is the picture of cross-linked group of Example 3 took after culturing in vitro for 6 days, e-2 is the picture of cross-linked group of Example 3 took after culturing in vitro for 15 days, f-1 is the picture of cross-linked group of Example 4 took after culturing in vitro for 15 days, f-2 is the picture of cross-linked group of Example 4 took after culturing in vitro for 24 days.

### DETAILED DESCRIPTION OF THE EMBODIMENTS

The present disclosure is further described with the following specific examples.

### Example 1: Preparation Method of Cross-Linked Collagen Melanocyte Complex

1 g collagen sponge was cut into 1 cm*1 cm squares, put into a 1000 mL beaker. 500 mL of 90% ethanol was added, stirred magnetically at room temperature. 9.6 g EDC and 11.5 g NHS were added, and then reacted for 48 h. After the reaction, the cross-linked collagen sponge was soaked with a 0.1 mol/L phosphate buffer solution, stirred magnetically for 1 h, washed with purified water, freeze-dried and sterilized by irradiation.

The sterile collagen sponge was cut into materials of uniform size: the upper surface being 1 cm*1 cm square, and the thickness being 5 mm. The cutting scaffold material was then pre-plated onto a cell culture plate. 200 μl melanocytes were inoculated on a collagen scaffold at a density of 1*10⁵ cells/ml, and cultured in a cell incubator at 37° C., 5% CO₂, and saturated humidity. After 2 hours of culture, 1 ml cell culture medium (without penicillin streptomycin) was added to the culture plate plated with the scaffold material, and the cells were cultured in the cell incubator at 37° C., 5% CO₂, and saturated humidity. The cell medium was changed every two days and the culture time was 11 days.

### Example 2: Preparation Method of Cross-Linked Collagen Melanocyte Complex

1 g collagen sponge was cut into 1 cm*1 cm squares, put into a 1000 mL beaker. 500 mL of 90% ethanol was added, stirred magnetically at room temperature. 9.6 g EDC and 11.5 g NHS were added, and then reacted for 48 h. After the reaction, the cross-linked collagen sponge was soaked with a 0.1 mol/L phosphate buffer solution, stirred magnetically for 1 h, washed with purified water, freeze-dried and sterilized by irradiation.

The sterile collagen sponge was cut into materials of uniform size: the upper surface being 1 cm*1 cm square, and the thickness being 5 mm. The cutting scaffold material was then pre-plated onto a cell culture plate. 400 μl melanocytes were inoculated on a collagen scaffold at a density of 1*10⁵ cells/ml, and cultured in a cell incubator at 37° C., 5% CO₂, and saturated humidity. After 2 hours of culture, 1 ml cell culture medium (without penicillin streptomycin) was added to a collagen plate-covered culture plate, and the cells were cultured in the cell incubator at 37° C., 5% CO₂, and saturated humidity. The cell medium was changed every two days and the culture time was 16 days.

### Example 3: Preparation Method of Cross-Linked Collagen Melanocyte Complex

1.5 g collagen sponge wan cut into 1 cm*1 cm squares, put into a 1000 mL beaker, 500 mL of 90% ethanol was added, stirred magnetically at room temperature, 9.6 g EDC and 11.5 g NHS were added, and then reacted for 48 h. After the reaction, the cross-linked collagen sponge was soaked with a 0.1 mol/L phosphate buffer solution, stirred magnetically for 1 h, washed with purified water, freeze-dried and sterilized by irradiation.

The sterile collagen sponge was cut into materials of uniform size: the upper surface being 1 cm*1 cm square, and the thickness being 5 mm. The cutting scaffold material was then pre-plated onto a cell culture plate. 400 μl melanocytes were inoculated on a collagen scaffold at a density of 1*10⁵ cells/ml, and cultured in a cell incubator at 37° C., 5% CO₂, and saturated humidity. After 2 hours of culture, 1 ml cell culture medium (without penicillin streptomycin) was added to the culture plate plated with the scaffold material, and the cells were cultured in the cell incubator at 37° C., 5% CO₂, and saturated humidity. The cell medium was changed every two days and the culture time was 16 days.

### Example 4: Preparation Method of Cross-Linked Collagen Melanocyte Complex

0.5 g collagen sponge was cut into 1 cm*1 cm squares, put into a 1000 mL beaker, 500 mL of 90% ethanol was added, stirred magnetically at room temperature, 7.68 g EDC and 4.6 g NHS were added, and then reacted for 48 h. After the reaction, the cross-linked collagen sponge was soaked with a 0.1 mol/L phosphate buffer solution, stirred magnetically for 1 h, washed with purified water, freeze-dried and sterilized by irradiation.

The sterile collagen sponge was cut into materials of uniform size: the upper surface being 1 cm*1 cm square, and the thickness being 5 mm. The cutting scaffold material was then pre-plated onto a cell culture plate. 200 μl melanocytes were inoculated on a collagen scaffold at a density of 2.5*10⁵ cells/ml, and cultured in a cell incubator at 37° C., 5% CO₂, and saturated humidity. After 2 hours of culture, 1 ml cell culture medium (without penicillin streptomycin) was added to the culture plate plated with the scaffold material, and the cells were cultured in the cell incubator at 37° C., 5% CO₂, and saturated humidity. The cell medium was changed every two days and the culture time was 24 days.

### Example 5: Preparation Method of Uncross-Linked Collagen Melanocyte Complex

The treated bovine Achilles tendon was dissolved with dilute acid, digested with pepsin for 72-90 hours, NaCl was added to a concentration of 2.5˜2.6 mol·L⁻¹, filtered and salt out for more than 24 hours, PBS solution dialyzed, freeze-dried into a sponge shape, and radiation sterilization after cut.

The sterile uncross-linked collagen sponge was cut into materials of uniform size: the upper surface being 1 cm*1 cm square, and the thickness being 5 mm. The cutting scaffold material was then pre-plated onto a cell culture plate. 200p1 melanocytes were inoculated on a collagen scaffold at a density of 2.5*10⁵ cells/ml, and cultured in a cell incubator at 37° C., 5% CO₂, and saturated humidity. After 2 hours of culture, 1 ml cell medium (without penicillin streptomycin) was added to the culture plate plated with the scaffold material, and the cells were cultured in the cell incubator at 37° C., 5% CO₂, and saturated humidity. The cell medium was changed every two days and the culture time was 15 days.

Result Analysis:

1) Analysis of Mechanical Properties of Cross-Linked Collagen Scaffold Materials

Mechanical property testing method: both ends of the scaffold material are held with a clip and pulled until the material breaks. Test conditions: the tensile speed is 300 mm/min, the humidity is 57%, the maximum peeling force is measured with a tensile machine and is the average of three measurements. The maximum peeling forces of the cross-linked collagen scaffold materials prepared in Examples 1, 2, 3, and 4 are all above 2.5N. This indicates that the cross-linked collagen scaffold material prepared by this method has good mechanical properties, and ensures that the collagen scaffold material can be freely sheared without breaking during the process of using according to requirements. After the culture in vitro of the cross-linked collagen melanocyte complex in Examples 1, 2, 3, and 4 for 11-24 days, no significant degradation or fragmentation of the scaffold material is found, and the material remained intact like starting to inoculate melanocytes, and the largest peeling force is still above 2.5N. At the same time, the collagen melanocyte complex cultured in vitro can be successfully transferred to the affected area without breaking.

2) Cytotoxicity Analysis of Cross-Linked Collagen Scaffold Material

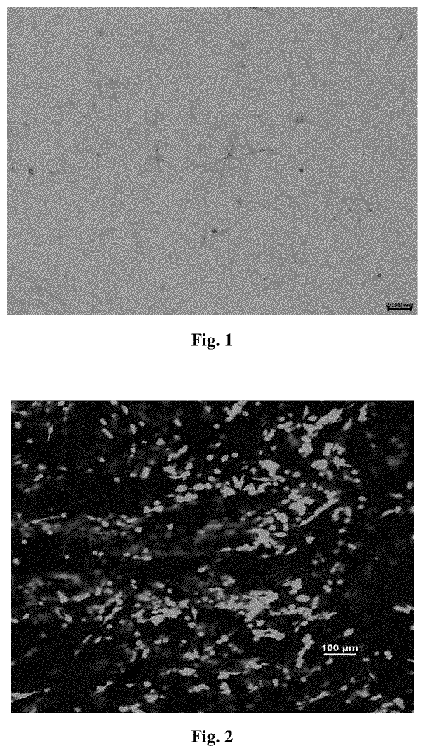

The CCK-8 cytotoxicity test of the cross-linked collagen scaffolds prepared in Examples 1, 2, 3, and 4 shows that the survival rate of the cell cultured in scaffold extracts prepared by this method is above 97%. The morphology of the cells is normal, the cells are slender, the number of dendrites is in a range of 2 to 5 or more, and different dendrites have different lengths (as shown in FIG. 1), which is not significantly different from that of the blank group. It shows that the collagen material prepared by this method has good biocompatibility and can be applied in tissue engineering and biomedical fields.

3) Cell Proliferation Analysis

The cross-linked collagen melanocyte complex in Example 4 was cultured for 15 days. After fluorescence (green) staining of live cells, laser confocal observation reveals that the melanocytes are proliferated a lot in the cross-linked collagen scaffold. There are more living melanocytes on the surface of the scaffold material and inside the three-dimensional structure. This shows that the three-dimensional structure of the cross-linked collagen scaffold material prepared by this method is beneficial to the growth of melanocytes.

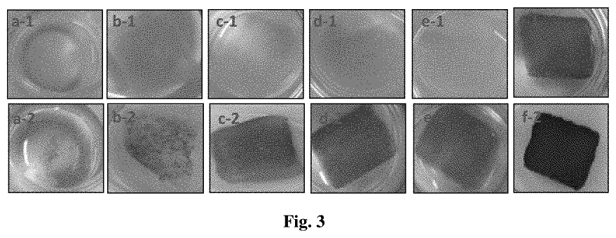

The results of the cell culture in vitro of Examples 1, 2, 3, and 4 are shown in FIG. 3. By observing FIG. 3, it is found that the melanocytes did not proliferate significantly in the uncross-linked group in 6 days; the melanocytes proliferated significantly in 15 days, and the material also became significantly darker due to melanin secretion. However, the thickness of the material had been degraded from 5 mm to approximately 0.5 mm, which was a thin layer. In the cross-linked group, the melanocyte proliferated significantly and the melanin secreted significantly. A large amount of melanin secreted in 24 days, and the scaffold material had changed from light black to dark black. This phenomenon indicates that the melanocytes have higher cell activity on the scaffold material. The scaffold material also provides a good three-dimensional growth environment for the cells, which is conducive to the proliferation of the cells in three-dimensional space. The amounts of melanin secreted by the collagen melanocytes obtained at different concentrations or different culture conditions are different, and it is expected that different skin colors can be adjusted accordingly.

The above description is only part of the embodiments of the present disclosure, and the protection scope of the present disclosure is not limited to the above embodiments, and does not represent all technical schemes under the concept of the present disclosure. It should be pointed out that, for those skilled in the art, any improvement and retouching should be considered as the scope of protection of the present disclosure if it is inspired by the concept of the patent and specific implementation cases without departing from the principle of the present disclosure.

1. A preparation method for a collagen melanocyte complex, comprising preparing a cross-linked collagen sponge scaffold material firstly, inoculating melanocytes on the cross-linked collagen sponge scaffold material and then culturing in vitro.

2. The preparation method according to claim 1, wherein a method for preparing the cross-linked collagen sponge scaffold material comprises the following steps: adding a collagen with appropriate size to a reaction solvent, adding a cross-linking agent to the reaction solvent, and magnetically stirring; after completion of the reaction, performing washing, freeze-drying, and sterilizing.

3. The preparation method according to claim 1, wherein the collagen is aquatic animal collagen or mammalian collagen; the collagen has a thickness of 0.5-10 mm sponge, which is cut into a shape not larger than a petri dish; the reaction solvent is ethanol or water phase, preferably 90% ethanol or PBS solution with pH=5.5; the cross-linking agent is EDC and NHS, and a molar ratio of EDC:NHS is in a range of 1:1 to 1:4; a time of the magnetic stirring is in a range of 2-72 h, preferably in a range of 24-48 h; a reaction temperature is in a range of 4-30° C., preferably in a range of 20-25° C.; and the washing condition includes soaking in phosphate buffer for 1-2 h, and washing with purified water for 1-24 h.

4. The preparation method according to claim 1, wherein inoculating melanocytes on the cross-linked collagen sponge scaffold material and culturing in vitro comprise the following steps: pre-plating the collagen sponge scaffold material onto a cell culture plate, inoculating 100 μl-1000 μl melanocytes to the collagen sponge scaffold material at a density of 2*10⁴ to 2*10⁷ cells/ml, and then culturing in a cell incubator under the condition of 37° C., 5% CO₂, and saturated humidity; after the collagen scaffold inoculated with melanocytes is cultured for 30 min-2 h, supplementing with cells medium 1 ml-10 ml, and continuing culturing in a cell incubator under the condition of 37° C., 5% CO₂, and saturated humidity; replacing the cell medium of the culture plate covered with the collagen sponge scaffold material with a new cell medium every two days, a culture time being in a range of 1 to 25 days, and obtaining the melanocyte-collagen complex.

5. The preparation method according to claim 1, wherein the melanocytes are human melanocytes; and the human melanocytes are melanocytes extracted from autologous or allogeneic tissues.

6. The preparation method according to claim 1, wherein skin color is adjusted by adjusting the density, number or culture time of the inoculated cells.

7. A collagen melanocyte complex, wherein the collagen melanocyte complex is a complex of a collagen scaffold and a melanocyte prepared by the method of claim 1.

8. A method for treating pigment deficiency and epidermal damage, comprising administering to a subject in need thereof a therapeutically effective amount of the collagen melanocyte complex of claim 7.

9. The method according to claim 8, wherein when preparing the collagen melanocyte complex, the skin color is adjusted by adjusting the density, number or culture time of the inoculated cells.
